(19) United States
(12) Patent Application Publication
(10) Pub. No.: US 2023/0129899 A1
(43) Pub. Date: Apr. 27, 2023

(54) COMPOUND, PHOTOSENSITIZER COMPRISING SAME, COMPOSITION FOR DIAGNOSING OR TREATING TUMOR TARGETING MITOCHONDRIA, AND PHOTODYNAMIC TREATMENT METHOD USING COMPOSITION

(71) Applicant: KYTECBIO CO., LTD., Seoul (KR)

(72) Inventors: Ju Young YOON, Seoul (KR); Van Nghia NGUYEN, Seoul (KR); Na Hyun KWON, Seoul (KR); Sujie QI, Seoul (KR)

(73) Assignee: KYTECBIO CO., LTD., Seoul (KR)

(21) Appl. No.: 17/911,647

(22) PCT Filed: Mar. 16, 2021
(86) PCT No.: PCT/KR2021/003242

§ 371 Date: Sep. 14, 2022

(30) Foreign Application Priority Data
Mar. 17, 2020 (KR) 10-2020-0032872
May 18, 2020 (KR) 10-2020-0059335

(51) Int. Cl. C07F 5/02; A61K 49/00

(52) U.S. Cl. CPC C07F 5/022; A61K 49/0052

(57) ABSTRACT

Disclosed are a compound, a photosensitizer including the compound, a composition for mitochondria-targeted diagnosis or therapy of tumors including the compound, and a photodynamic therapy using the composition. The compound may be represented by Formula 1:

### TECHNICAL FIELD

The present disclosure relates to a compound, a photosensitizer comprising the compound, a composition for mitochondria-targeted diagnosis or therapy of tumors comprising the compound, and a photodynamic therapy using the composition.

### BACKGROUND ART

Photodynamic therapy (PDT) may be an effective clinical treatment strategy for malignant tumors. In the process of photodynamic therapy, a photosensitizer (PS) is activated under light irradiation, and the excited photosensitizer interacts with oxygen molecules to generate cytotoxic reactive oxygen species (ROS) and can oxidize biomolecules to destroy cancer cells.

Examples of such photodynamic therapy agents include numerous organic dyes such as porphyrin, phthalocyanine, cyanine, squaraine, diketopyrrolopyrrole (DPP), boron dipyrromethane (BODIPY), and the like. Among such organic dyes, boron dipyrromethane (BODIPY) has garnered attention in photodynamic therapy due to its excellent photochemical stability, good biocompatibility, high molar extinction coefficients, and excellent quantum efficiency of fluorescence.

Presently, most boron dipyrromethane (BODIPY) organic dyes introduce heavy halogen atoms such as Br and I into the core, in order to enhance spin-orbit coupling. However, the introduction of such heavy halogen elements into the core gives rise to issues of increasing dark-toxicity and quench fluorescence.

To address such issues, there is a demand for a novel compound that has no heavy halogen elements introduced into the core, a photosensitizer including the compound, a composition for mitochondria-targeted diagnosis or treatment of tumors including the compound, and a photodynamic therapy using the composition.

### DESCRIPTION OF EMBODIMENTS

### Technical Problem

One aspect provides a novel compound with no heavy atom introduced therein.

Another aspect provides a photosensitizer for photodynamic therapy, including the compound.

Another aspect provides a composition for mitochondria-targeted diagnosis or therapy of tumors, including the compound as an active ingredient.

Another aspect provides a photodynamic therapy method using the composition.

### Solution to Problem

According to one aspect, a compound represented by Formula 1 below is provided:

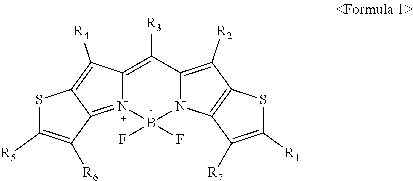

In Formula 1,

R₁ and R₅ may be each independently hydrogen, a substituted or unsubstituted C1-C20 alkyl group, a substituted or unsubstituted C6-C30 aryl group, or a combination thereof;

R₂, R₄, R₆, and R₇ may be each independently hydrogen, a substituted or unsubstituted C1-C20 alkyl group, or a combination thereof; and

R₃ may be a substituted or unsubstituted C6-C30 aryl group, a substituted or unsubstituted C6-C30 heteroaryl group, or a combination thereof.

According to another aspect, a photosensitizer including the above-described compound may be provided.

The photosensitizer may generate reactive oxygen species by irradiation of light in a wavelength range of 450 nm to 800 nm.

The photosensitizer may be for fluorescence imaging and mitochondria-targeted photodynamic therapy.

According to another aspect, provided is a composition for mitochondria-targeted diagnosis or treatment of tumors, including the above-described compound and a pharmaceutically acceptable salt thereof as an active ingredient.

The composition may include the compound or supramolecules of a pharmaceutically acceptable salt thereof.

The mean size of the supramolecules may be 1 nm to 200 nm.

The tumor may be selected from among breast cancer, kidney cancer, testicular cancer, prostate cancer, ovarian cancer, uterine cancer, cervical cancer, vaginal cancer, fallopian tube cancer, rectal cancer, lung cancer, stomach cancer, liver cancer, esophageal cancer, small intestine cancer, pancreatic cancer, oral cancer, melanoma, or sarcoma.

According to another aspect, there may be provided a photodynamic therapy method including: contacting a non-human mammalian subject with the above-described composition for diagnosis or treatment of tumor;

allowing a time for the composition for diagnosis or treatment of tumor to be distributed within a target cell; and

irradiating a target cell area in the subject with light.

### Advantageous Effects of Disclosure

A compound represented by Formula 1 according to one aspect may provide a novel compound having no heavy atoms introduced therein. A photosensitizer including the compound may generate reactive oxygen species by light irradiation and may be utilized in fluorescence imaging and mitochondria-targeted photodynamic therapy. A composition including the compound as an active ingredient may be used in mitochondria-targeted diagnosis or treatment of tumors. There may be provided a photodynamic therapy method using a composition including the compound.

### BRIEF DESCRIPTION OF DRAWINGS

FIG. 1 is a schematic diagram showing S₁-T₁ intersystem crossing (ISC) based on charge-transfer and reactive oxygen species generation of a compound according to an embodiment.

FIG. 2 shows results of analysis of UV-vis absorption spectrums of PY-BOD (Compound 1), PH-BOD (Compound 2), MeO-BOD (Compound 3), and DMA-BOD (Compound 4) obtained in Synthesis Examples 1-4.

FIG. 3 shows results of analysis of fluorescence spectrums of PY-BOD (Compound 1), PH-BOD (Compound 2), MeO-BOD (Compound 3), and DMA-BOD (Compound 4) obtained in Synthesis Examples 1-4.

FIG. 4 shows results of analysis of time-dependent photodegradation of 1,3-diphenylisobenzofuran (DPBF) in the presence of a sample solution prepared by dissolving MeO-BOD (Compound 3) obtained by Synthesis Example 3 in acetonitrile to have an absorbance of 0.2 at 560 nm, under 560 nm light irradiation at 0.1 W/cm² for 0 seconds, 2 seconds, 4 seconds, 6 seconds, 8 seconds, 10 seconds, 12 seconds, 14 seconds, and 16 seconds.

FIG. 5 shows results of analysis of photodegradation of 1,3-diphenylisobenzofuran (DPBF) in the presence of 1,3-diphenylisobenzofuran (DPBF, control), PY-BOD (Compound 1), PH-BOD (Compound 2), MeO-BOD (Compound 3), and DMA-BOD (Compound 4) obtained in Synthesis Examples 1-4, and rose bengal, in acetonitrile.

FIG. 6 shows TEM images of 5.0 μM MeO-BOD (Compound 3) supramolecules obtained by dropwise addition of dimethyl sulfoxide (DMSO) stock solution of MeO-BOD (Compound 3) obtained in Synthesis Example 3 to 3.0 mL of distilled water.

FIG. 7 shows results of analysis of a UV-vis absorption spectrum of the MeO-BOD (Compound 3) supramolecules.

FIG. 8 shows results of cell imaging performed by a laser confocal microscope on MeO-BOD (Compound 3) supramolecules obtained in FIG. 6 at a concentration of 1.0 μM after being cultured together with HeLa cells for 0 minutes, 15 minutes, 30 minutes, and 60 minutes.

FIG. 9 shows results of cell imaging performed by a laser confocal microscope on 1.0 μM MeO-BOD (Compound 3) in FIG. 8 after being cultured together with HeLa cells and further stained with LysoTracker Green DND 26.

FIG. 10 shows results of cell imaging performed by a laser confocal microscope on 1.0 μM MeO-BOD (Compound 3) in FIG. 8 after being cultured together with HeLa cells and further stained with MitoTracker Green FM (500 nM).

FIG. 11 shows a correlation between fluorescence intensity and 1.0 μM MeO-BOD (Compound 3) supramolecules obtained in FIG. 6 after incubation and staining according to FIG. 9.

FIG. 12 shows a correlation between fluorescence intensity and 1.0 μM MeO-BOD (Compound 3) obtained in FIG. 6 after incubation and staining according to FIG. 10.

FIG. 13 shows results of cell viability analysis using an MTT assay after continuous incubation of the cells in a dark room for 24 hours.

FIG. 14 shows results of cell viability analysis using an MTT assay after continuous incubation of cells continuously for 24 hours using 560 nm light of a halogen lamp (0.1 W/cm²) for different light irradiation times (0, 5, and 10 minutes).

FIG. 15 shows results of analysis of fluorescence images of Calcein AM/PI-stained HeLa cells after irradiation with 560 nm light (0.1 W/cm², 5 minutes or 10 minutes) with respect to HeLa cells cultured with addition of 0.5 μM and 10.0 μM MeO-BOD (Compound 3) obtained in Synthesis Example 3.

FIG. 16 shows fluorescence images of ROS generated in HeLa cells using 10 μM of 2,7-dichlorofluorescein diacetate (DCFH-DA) as an indicator after incubation with 1.0 μM MeO-BOD (Compound 3) supramolecules obtained in Analysis Example 2.

FIG. 17 shows fluorescence images of the mitochondrial membrane potential of HeLa cells using 2 μM JC-1 as an indicator after incubation with or without 1.0 μM MeO-BOD (Compound 3) supramolecules obtained in Analysis Example 2.

### MODE OF DISCLOSURE

Hereinafter, with reference to the accompanying drawings, a compound according to an embodiment of the present disclosure, a photosensitizer including the compound, a composition for mitochondria-targeted diagnosis or treatment of tumor, including the compound, and a photodynamic treatment method using the composition will be described in greater detail. Hereinbelow, the present disclosure is not limited by the following examples but rather, the present disclosure is only defined by the scope of the claims to be described below.

The terms “comprise(s)” and/or “comprising,” as used herein, unless otherwise specified, does not preclude the presence or addition of one or more other elements.

As used herein, the term “a combination thereof” refers to a mixture or combination of one or more of the described components.

As used herein, “pharmaceutically acceptable salt” refers to any organic or inorganic addition salt of a compound such as one indicated by Formula 1, etc. that side effects attributable to the salt do not degrade benefits of the compound represented by Formula 1, etc. at a concentration providing a beneficial effect that is relatively nontoxic and harmless to a patient. For these salts, inorganic acids and organic acids can be used as free acids, and such inorganic acids include hydrochloric acid, hydrobromic acid, nitric acid, sulfuric acid, perchloric acid, phosphoric acid, and the like, and such organic acids include citric acid, acetic acid, lactic acid, maleic acid, fumaric acid, gluconic acid, methanesulfonic acid, glycolic acid, succinic acid, tartaric acid, galacturonic acid, embonic acid, glutamic acid, aspartic acid, oxalic acid, (D) or (L) malic acid, maleic acid, methanesulfonic acid, ethanesulfonic acid, 4-toluenesulfonic acid, salicylic acid, citric acid, benzoic acid, malonic acid, or the like. Further, these salts include alkali metal salts (sodium salt, potassium salt, etc.) and alkaline earth metal salts (calcium salt, magnesium salt, etc.) and the like. For example, such acid addition salts may include acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, bibenzate, hydrochloride/chloride, hydrobromide/bromide, hydroiodide/iodide, isethionate, lactate, malate, maleate, malonate, mesylate, methyl sulfate, naphthylate, 2-naphthylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, zinc salts, and the like. In particular, such acid addition salts may be hydrochloride or trifluoroacetate.

As used herein, the term “supramolecules” refers to a set of molecules generated, without bonding to other molecules, but by gathering of two or more smaller molecules through intermolecular bonding or attraction, such as hydrogen bonding, electrostatic interaction, van der Waals attraction, etc., without binding to other molecules.

A compound according to an embodiment may be represented by Formula 1:

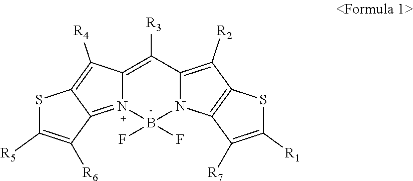

In Formula 1,

R₁ and R₅ may be each independently hydrogen, a substituted or unsubstituted C1-C20 alkyl group, a substituted or unsubstituted C6-C30 aryl group, or a combination thereof;

R₂, R₄, R₆, and R₇ may be each independently hydrogen, a substituted or unsubstituted C1-C20 alkyl group, or a combination thereof; and

R₃ may be a substituted or unsubstituted C6-C30 aryl group, a substituted or unsubstituted C6-C30 heteroaryl group, or a combination thereof.

For example, the substituted or unsubstituted C6-C30 aryl group in R₃ may include a C6-C30 aryl group unsubstituted or substituted with —CHO, —ORₐ, —NRₐ, —NHCORₐ, or —OCORₐ, wherein Rₐ may be hydrogen, a C1-C20 alkyl group, or a C6-C20 aryl group.

For example, the substituted or unsubstituted C6-C30 heteroaryl group in R₃ may include a pyridyl group, a pyrrolopyridyl group, a pyrazolopyridyl group, a thienopyridyl group, a pyrimidyl group, a pyrazolyl group, a pyrrolyl group, an imidazolyl group, an indolyl group, an indenyl group, a quinolyl group, or a thiophenyl group.

For example, the compound may include a compound represented by Formula 2:

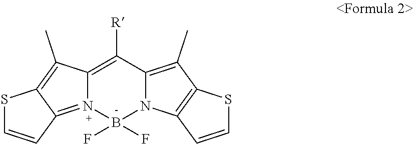

In Formula 2,

R′ may be a substituted or unsubstituted C6-C30 aryl group, a substituted or unsubstituted C6-C30 heteroaryl group, or a combination thereof.

For example, the substituted or unsubstituted C6-C30 aryl group of R′ may include a C6-C30 aryl group unsubstituted or substituted with —CHO, —OR′ₐ, —NR′ₐ, —NHCOR′ₐ, —COOR′ₐ, —C₆H₅COOR′ₐ, or —OCOR′ₐ, and R′ₐ may be hydrogen, a C1-C20 alkyl group, or a C6-C20 aryl group.

For example, the compound may include Compounds 1 to 4:

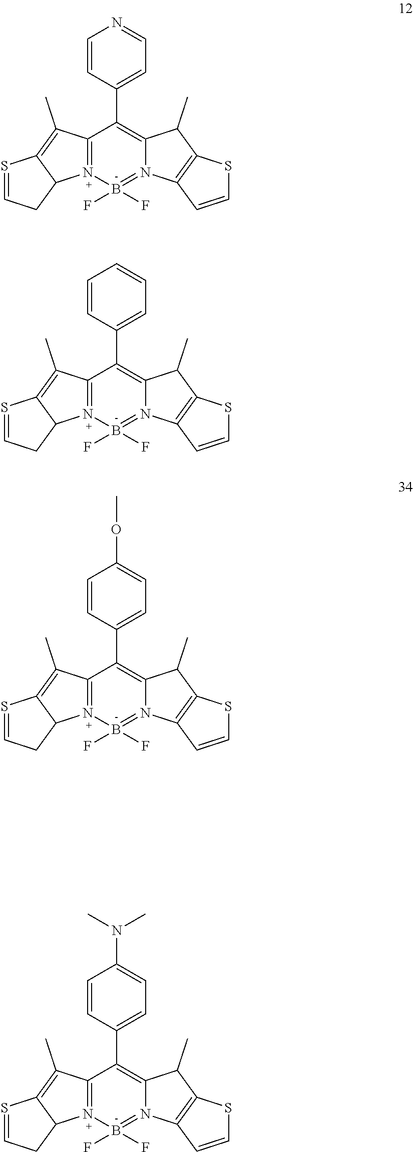

The compound represented by Formula 1 has a structure that has a strong electron donor introduced at meso-site of thiophene-boron dipyrromethene (thiophene-BIODIPY) core, without introducing heavy elements, e.g. heavy halogen atoms, into the core. Due to such structural characteristics, the compound represented by Formula 1 can overcome issues associated with toxicity, lack of degradability, and costs, and thus is suitable for biological applications, and furthermore, through Type II process, can enhance ¹O2 generation capability and be used as an agent for mitrochondria-targeted diagnosis or photodynamic therapy.

The photosensitizer according to another embodiment may include the above-described compound.

The photosensitizer may generate reactive oxygen species by irradiation of light in a wavelength range of 450 nm to 800 nm.

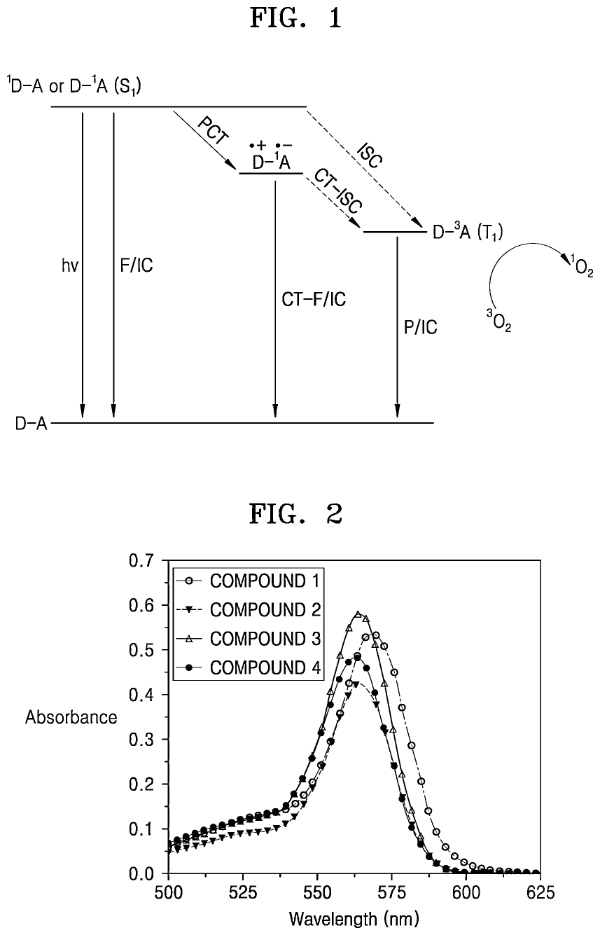

FIG. 1 is a schematic diagram showing S₁-T₁ intersystem crossing (ISC) based on charge-transfer and generation of reactive oxygen species of a compound according to an embodiment. Here, D is an electron donor, A is an electron acceptor, IC represents internal conversion, PCT represents photo-induced charge transfer, CT represents charge transfer, ISC represents intersystem crossing, P represents phosphorescence, S₁ represents a first singlet excited state, and T₁ represents a first triplet excited state.

Referring to FIG. 1, a compound according to an embodiment may, for example, enhance spin-orbit coupling by reducing singlet energy gaps when irradiated with light in a wavelength range of 450 nm to 800 nm, and enhance S₁-T₁ intersystem crossing (ISC) to thereby enhance reactive oxygen species (¹O₂) generation capacity. This is considered to be attributable to the structure that has strong electron donors introduced at meso-site of the thiophene-BODIPY core.

The photosensitizer may target fluorescence imaging and mitochondria.

A composition for mitochondria-targeted diagnosis or treatment of tumors according to another embodiment may include the above-described compound or a pharmaceutically acceptable salt thereof as an active ingredient.

The composition may be prepared by adding the above-described compound to a solvent, a buffer solution, or a mixture thereof, and further adding an acid and/or a base thereto. In addition, the composition may further include other additives which may be used in the relevant technical field. The amounts of the solvent, acid, base, and buffer solution included in the composition may be appropriately adjusted depending on a required performance. In addition, the composition may be mixed with a sample. The sample may be a biological sample including one or more selected from among a microorganism, a cell, and a tissue, but is not necessarily limited thereto, and may be any sample that can be used as a biological sample in the art.

The composition may include the compound or supramolecules of a pharmaceutically acceptable salt thereof.

For example, the mean size of the supramolecules may be 1 nm to 200 nm. For example, the mean size of the supramolecules may be from 5 nm to 190 nm, 10 nm to 180 nm, 20 nm to 170 nm, or 30 nm to 150 nm.

The tumor may be selected from among breast cancer, kidney cancer, testicular cancer, prostate cancer, ovarian cancer, uterine cancer, cervical cancer, vaginal cancer, fallopian tube cancer, rectal cancer, lung cancer, stomach cancer, liver cancer, esophageal cancer, small intestine cancer, pancreatic cancer, oral cancer, melanoma, or sarcoma.

A photodynamic therapy method according to another embodiment may include: contacting a non-human mammalian subject with the above-described composition for diagnosis or treatment of tumors; allowing a time for the composition for diagnosis or treatment of tumors to be distributed within a target cell; and irradiating a target cell area in the subject.

The photodynamic therapy method may have benefits, such as time and space selectivity, non-invasiveness, and reduction of adverse side effects.

In this specification, substituents are induced by replacement of one or more hydrogen atoms in an unsubstituted mother group with other atoms or functional groups. Unless otherwise stated, when a functional group is considered to be “substituted,” it means that the functional group is substituted with at least one substituent selected from among an alkyl group having 1 to 40 carbon atoms, an alkenyl group having 2 to 40 carbon atoms, an alkynyl group having 2 to 40 carbon atoms, a cycloalkyl group having 3 to 40 carbon atoms, a cycloalkenyl group having 3 to 40 carbon atoms, and an aryl group having 7 to 40 carbon atoms. When a functional group is described as being “optionally substituted”, it is meant that the functional group may be substituted with the aforementioned substituents.

In this specification, a and b in “a to b carbon atoms” refers to the number of carbon atoms of a certain functional group. That is, the functional group may include from a to b carbon atoms. For example, “an alkylene group having 1 to 4 carbon atoms” refers to an alkylene group that has 1 to 4 carbon atoms, e.g., —CH₂—, —CH₂CH₂—, —CH₂CH₂CH₂—, —(CH₃)₂C—, —CH₂CH₂CH₂CH₂—, —CH₂CH₂CH(CH₃)—, and —(CH₃)₂C—.

In this specification, the term “alkyl” refers to a branched or unbranched aliphatic hydrocarbon. In an embodiment, an alkyl group may be substituted, or unsubstituted. Examples of the alkyl group may include, without being necessarily limited to a methyl group, an ethyl group, a propyl group, an isopropyl group, a butyl group, an isobutyl group, tert-butyl group, a pentyl group, a hexyl group, a cyclopropyl group, a cyclopentyl group, a cyclohexyl group, a cycloheptyl group, and the like, and such an alkyl group may be optionally substituted or may be unsubstituted. In an embodiment, the alkyl group may contain 1 to 6 carbon atoms. For examples, an alkyl group having 1 to 6 carbon atoms may be methyl, ethyl, propyl, isopropyl, butyl, iso-butyl, sec-butyl, pentyl, 3-pentyl, hexyl, and the like, but is not necessarily limited thereto.

In this specification, the term “alkenyl” refers to a branched or unbranched hydrocarbon having at least one carbon-carbon double bond. Non-limiting examples of an alkenyl group include vinyl, allyl, butenyl, isopropenyl, isobutenyl, and the like.

In this specification, the term “alkynyl” refers to a branched or unbranched hydrocarbon having at least one carbon-carbon triple bond. Non-limiting examples of an alkynyl group include ethynyl, butynyl, isobutynyl, isopropynyl, and the like.

In this specification, the term “cycloalkyl” refers to a monovalent saturated monocyclic hydrocarbon. Non-limiting examples of the cycloalkyl group include a cyclopropyl group, a cyclobutyl group, a cyclopentyl group, a cyclohexyl group, a cycloheptyl group, and the like.

As used herein, the term “heterocycloalkyl” refers to a monovalent monocyclic hydrocarbon that contains at least one hetero atom selected from N, O, Si, P and S as a ring-forming atom. Non-limiting examples of the heterocycloalkyl group include 1,2,3,4-oxatriazolidinyl group, a tetrahydrofuranyl group, a tetrahydrothiophenyl group, and the like.

As used herein, the term “cycloalkenyl” is a monovalent monocyclic hydrocarbon that contains at least one double bond in the ring or has no aromaticity. Non-limiting examples of the cycloalkenyl group include a cyclopentenyl group, a cyclohexenyl group, a cycloheptenyl group, and the like.

As used herein, the term “heterocycloalkenyl” refers to a monovalent monocyclic hydrocarbon that contains at least one hetero atom selected from N, O, Si, P and S as a ring-forming atom, and contains at least one double bond in the ring. Non-limiting examples of the heterocycloalkenyl group include 4,5-dihydro-1,2,3,4-oxatriazolyl group, 2,3-dihydrofuranyl group, 2,3-dihydrothiophenyl group, and the like.

As used herein, the term “aryl” refers to an aromatic ring that only contains carbon atoms in the ring structure, a ring system (i.e., two or more fused rings sharing two adjacent carbon atoms), or a ring in which multiple aromatic rings are interconnected via a single bond, —O—, —S—, —C(═O)—, —S(═O)₂—, —Si(Rₐ)(R_{b})— (Rₐ and R_{b} are each independently an alkyl group having 1 to 10 carbon atoms), an alkylene group having 1 to 10 carbon atoms that is unsubstituted or substituted with halogen, or —C(═O)—NH—. If the aryl group is a ring system, then each ring in the system is aromatic. Examples of the aryl group include, but are not limited to a phenyl group, a biphenyl group, a naphthyl group, a phenalthrenyl group, a naphthacenyl group, and the like. The aryl group may be substituted or unsubstituted.

As used herein, the term “heteroaryl” refers to a monocyclic or bicyclic organic compound that contains one or more heteroatoms selected from N, O, P or S, wherein the remaining ring atoms are carbon. The heteroaryl group may include, for example, 1-5 heteroatoms, and may include 5-10 ring members. The S or N may be oxidized to have various oxidation states.

Non-limiting examples of “heteroaryl” include thienyl, furyl, pyrrolyl, imidazolyl, pyrazolyl, thiazolyl, isothiazolyl, 1,2,3-oxadiazolyl, 1,2,4-oxadiazolyl, 1,2,5-oxadiazolyl, 1,3,4-oxadiazolyl group, 1,2,3-thiadiazolyl, 1,2,4-thiadiazolyl, 1,2,5-thiadiazolyl, 1,3,4-thiadiazolyl, isothiazol-3-yl, isothiazol-4-yl, isothiazol-5-yl, oxazol-2-yl, oxazol-4-yl, oxazol-5-yl, isoxazol-3-yl, isoxazol-4-yl, isoxazol-5-yl, 1,2,4-triazol-3-yl, 1,2,4-triazol-5-yl, 1,2,3-triazol-4-yl, 1,2,3-triazol-5-yl, tetrazolyl, pyrid-2-yl, pyrid-3-yl, 2-pyrazin-2-yl, pyrazin-4-yl, pyrazin-5-yl, 2-pyrimidin-2-yl, 4-pyrimidin-2-yl, 5-pyrimidin-2-yl, or the like.

Examples and comparative examples of the present disclosure will be described below. However, the following examples are illustrative of the present disclosure, and therefore, the present disclosure is not limited thereto.

### EXAMPLES

All chemical agents were purchased from Sigma and used without further purification. MitoTracker DCFH-DA, Green FM, and LysoTracker Green DND-26 were purchased from Thermo (Invitrogen). Calcein-AM and PI were purchased from Sigma. Deuterated solvent from Cambridge Isotope Laboratories was used. All ¹H-NMR and ¹³C-NMR spectra were obtained using the Bruker AM 300 (300.13 MHz for ¹H and 75.48 MHz for ¹³C). Jeol JMS 700 high-resolution mass spectrometer was used to obtain FAB (fast atom bombardment) mass spectrums at Korea Basic Science Institute (Daegu). UV-visible absorption and fluorescence spectra were recorded using Thermo Scientific Evolution 201 UV-Visible Spectrophotometer and JASCO Spectrofluorometer FP-8500, respectively. Dynamic light scattering (DLS) was measured using Nano-ZS (Malvern). TEM images were obtained with JEOL-2100F electron microscope operating at 200 kV. Confocal laser scanning microscopic images were obtained using Olympus Fluoview FV1200 confocal laser scanning microscope.

Reaction Scheme 1 for the compounds prepared in Synthesis Examples 1 to 4 is as follows:

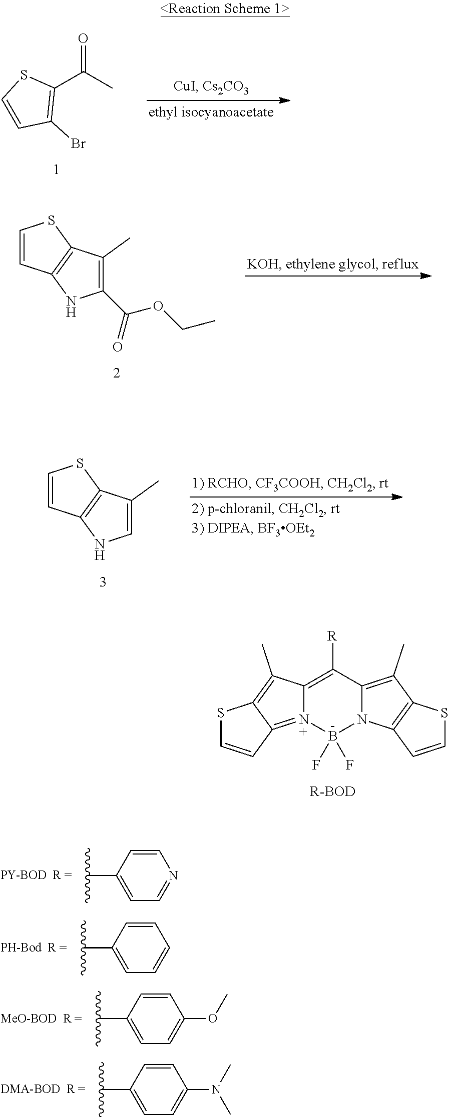

### Synthesis Example 1: PY-BOD Compound

A mixture of Compound 1 (1020 mg, 5 mmol) of Reaction Scheme 1, CuI (95 mg, 0.5 mmol), and Cs₂CO₃ (3.25 g, 10 mmol) was stirred in DMSO (3 mL) under N₂ atmosphere at room temperature for 30 minutes, and then ethyl isocyanoacetate (622 mg, 5.5 mmol) was dropwise added thereto. After stirring at 50° C. for 4 hours, the reaction mixture was extracted with CH₂Cl₂. The organic layer was washed twice with brine, and then the organic layer was dried over MgSO₄ and filtered. The filtrate was concentrated by evaporation, and the crude product was purified by column chromatography using a mixture of eluents, hexane and ethyl acetate (v/v=10:1) to obtain Compound 2 as white solid (559 mg, 53%).

Compound 2 (837 mg, 4 mmol) and potassium hydroxide (1.58 mg, 24 mmol) were suspended in ethylene glycol, and were heated to reflux under a N₂ atmosphere in the dark for 4 hours. The resulting reaction mixture was cooled to room temperature and then placed in cold water, followed by extraction with CH₂Cl₂. The organic layer was dried over MgSO₄ and then filtered. The filtrate was concentrated by evaporation and the crude product was purified via column chromatography using a mixed eluent (hexane and ethyl acetate=50:1) to produce Compound 3 as lemon yellow oil (310 mg, 56%).

Compound 3 (2 mmol, 2 equivalents) and PY-BOD aromatic aldehyde (1 mmol, 1 equivalent) were dissolved in dry CH₂Cl₂ (200 mL), and trifluoroacetic acid (6 drops) was added thereto, to produce a mixture. The mixture was stirred at room temperature for 18 hours. A portion of p-chloroaniline solution (1.2 equivalents) was added to CH₂Cl₂ (50 mL) and the resulting dark purple mixture was stirred for 30 minutes. Diisopropylethylamine (DIPEA, 18 equivalents) was added to this mixture and stirred for 30 minutes, and then BF₃.Et₂O (18 equivalents) was slowly added thereto. This mixture was stirred for 12 hours, and after adding water (100 mL) thereto, continued to be stirred for 1 hour. The reaction mixture was transferred to a separatory funnel and then washed with a portion of water. The organic layer was dried over MgSO₄ and the solvent was removed under reduced pressure. The organic layer was dried over MgSO₄ and the solvent was removed under reduced pressure. The crude product was purified via column chromatography using a mixed eluent (hexane and CH₂C12) to produce thiophene-fused PY-BOD pigment as purple-colored solid (17.5%).

¹H NMR (300 MHz, CDCl₃) δ 8.87 (d, J=5.6 Hz, 2H), 7.65 (d, J=5.3 Hz, 2H), 7.42 (dd, J=4.4, 1.6 Hz, 2H), 7.15 (d, J=5.3 Hz, 2H), 1.61 (s, 6H). ¹³C NMR (75 MHz, CDCl₃) δ 157.99, 151.10, 143.10, 141.75, 141.48, 136.82, 134.63, 133.68, 123.84, 114.48, 14.61. HRMS (ESI), calcd for (C₂₀H₁₄BF₂N₃NaS₂⁺): m/z [M+Na]⁺: 432.0588; found: m/z 432.0589.

### Synthesis Example 2: PH-BOD Compound

Thiophene-fused PH-BOD pigment as purple solid (7.1%) was obtained following the same method in Synthesis Example 1, except that PH-BOD aromatic aldehyde (1 mmol, 1 equivalent) instead of PY-BOD aromatic aldehyde (1 mmol, 1 equivalent) was dissolved in dry CH₂Cl₂ (200 mL) and trifluoroacetic acid (6 drops) was added to produce a mixture.

NMR (300 MHz, CDCl₃) δ 7.61 (d, J=5.3 Hz, 2H), 7.57 (dd, J=4.2, 2.4 Hz, 2H), 7.38 (dd, J=6.6, 3.0 Hz, 2H), 7.16 (d, J=5.3 Hz, 2H), 1.58 (s, 6H). ¹³C NMR (75 MHz, CDCl₃) δ 157.06, 145.72, 143.23, 140.32, 137.58, 134.04, 129.60, 129.45, 128.20, 118.85, 114.19, 14.02. HRMS (FAB⁺), calcd for (C₂₁H₁₅BF₂N₂S₂⁺): m/z [M]⁺: 408.0738; found: m/z 408.0739.

### Synthesis Example 3: MeO-BOD Compound

Thiophene-fused MeO-BOD pigment as purple solid (7.1%) was obtained following the same method in Synthesis Example 1, except that MeO-BOD aromatic aldehyde (1 mmol, 1 equivalent) instead of PY-BOD aromatic aldehyde (1 mmol, 1 equivalent) was dissolved in dry CH₂Cl₂ (200 mL) and trifluoroacetic acid (6 drops) was added to produce a mixture.

NMR (300 MHz, DMSO) δ 8.08 (d, J=5.3 Hz, 1H), 7.43 (d, J=8.6 Hz, 1H), 7.19 (d, J=8.7 Hz, 1H), 7.07 (d, J=5.3 Hz, 1H), 3.87 (s, 2H), 1.63 (s, 3H). ¹³C NMR (75 MHz, CDCl₃) δ 160.76, 157.07, 146.08, 140.27, 138.11, 134.13, 133.75, 129.68, 126.12, 114.98, 114.33, 55.55, 14.40. HRMS (FAB⁺), calcd for (C₂₂H₁₇BF₂N₂OS₂⁺): m/z [M]+: 438.0843; found: m/z 438.0846.

### Synthesis Example 4: DMA-BOD Compound

Thiophene-fused DMA-BOD pigment as purple solid (8.7%) was obtained following the same method in Synthesis Example 1, except that DMA-BOD aromatic aldehyde (1 mmol, 1 equivalent) instead of PY-BOD aromatic aldehyde (1 mmol, 1 equivalent) was dissolved in dry CH₂Cl₂ (200 mL) and trifluoroacetic acid (6 drops) was added to produce a mixture.

¹H NMR (300 MHz, CDCl₃) δ 7.58 (d, J=5.3 Hz, 2H), 7.20-7.10 (m, 4H), 6.89-6.73 (m, 2H), 3.07 (s, 6H), 1.71 (s, 6H). ¹³C NMR (75 MHz, CDCl₃) δ 156.65, 151.18, 147.72, 139.62, 138.41, 134.13, 133.50, 129.39, 121.02, 114.35, 112.47, 40.42, 14.59. HRMS (ESI), calcd for (C₂₃H₂₀BF₂N₃NaS₂⁺): m/z [M+Na]⁺: 474.1057; found: m/z 474.1059.

### Analysis Example 1: UV-Vis Absorption and Fluorescence Spectrums

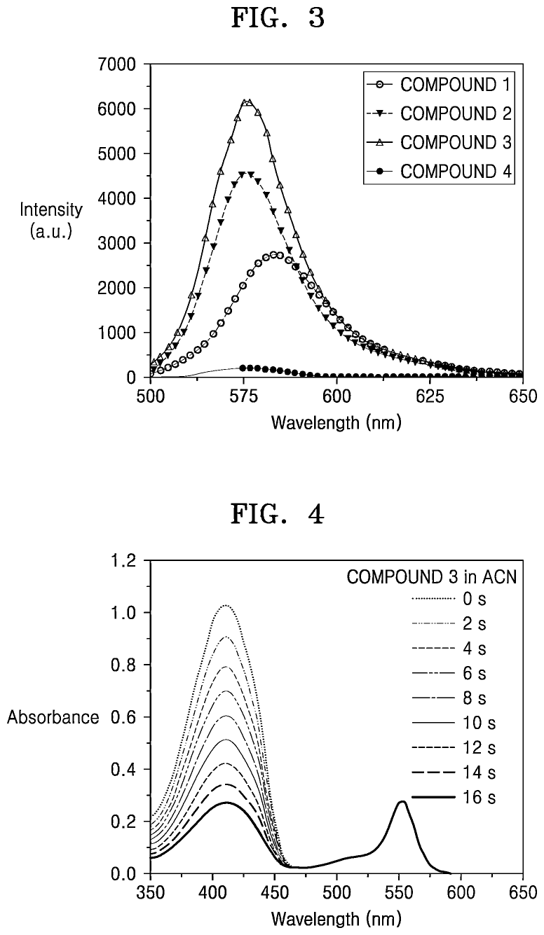

UV-vis absorption and fluorescence spectrums regarding PY-BOD (Compound 1), PH-BOD (Compound 2), MeO-BOD (Compound 3), and DMA-BOD (Compound 4) obtained in Synthesis Examples 1-4 were analyzed and shown in FIG. 2 and FIG. 3.

The UV-vis absorption and fluorescence spectrums were recorded using the Evolution 201 UV-vis spectrophotometer by Thermo Scientific and the FS-2 spectrometer (Scinco). Diluted sample solution (typically 5 μM) was prepared form a stock solution (1.0 mM) in ambient conditions. The UV-vis absorption and fluorescence spectrums were from an organic solution (5 μM) using 1 cm quartz cuvette.

Referring to FIG. 2, in the UV-vis absorption spectra, PY-BOD (Compound 1), PH-BOD (Compound 2), MeO-BOD (Compound 3), and DMA-BOD (Compound 4) obtained in Synthesis Examples 1-4 showed a maximum absorption wavelength (Aabs) of about 565 nm, showing a blueshift. This shows that PY-BOD (Compound 1), PH-BOD (Compound 2), MeO-BOD (Compound 3), and DMA-BOD (Compound 4) obtained in Synthesis Examples 1-4 may have a dipole moment of the ground state that is greater than the first excited singlet states thereof.

Referring to FIG. 3, in the fluorescence spectra, PY-BOD (Compound 1), PH-BOD (Compound 2), and MeO-BOD (Compound 3) showed a moderate fluorescence intensity. In particular, in the fluorescence spectra, DMA-BOD (Compound 4) showed a weak fluorescence intensity. From this result, it could be confirmed that the introduction of substituents may vary the fluorescence intensity of BOD compound and may result in a difference in singlet oxygen generation capability.

Regarding PY-BOD (Compound 1), PH-BOD (Compound 2), MeO-BOD (Compound 3), and DMA-BOD (Compound 4) obtained in Synthesis Examples 1 to 4, singlet oxygen generation capability was analyzed from UV-vis light absorption spectrums using 1, 3-diphenylisobenzofuran (DPBF, control) as an indicator.

Specifically, regarding a sample solution prepared by dissolving MeO-BOD (Compound 3) obtained by Synthesis Example 3 in acetonitrile to have an absorbance of 0.2 at 560 nm was irradiated with 560 nm light at 0.1 W/cm² for 0 second, 2 seconds, 4 seconds, 6 seconds, 8 seconds, 10 seconds, 12 seconds, 14 seconds, and 16 seconds, the analysis result of time-dependent photodegradation of 1,3-diphenylisobenzofuran (DPBF) are shown in FIG. 4.

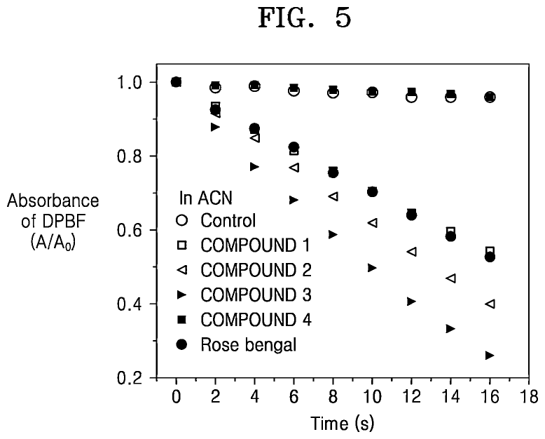

Additionally, regarding 1,3-diphenylisobenzofuran (DPBF, control), and PY-BOD (Compound 1), PH-BOD (Compound 2), MeO-BOD (Compound 3), and DMA-BOD (Compound 4) obtained in Synthesis Examples 1-4, and rose bengal in acetonitrile, the analysis result of time-dependent photodegradation of 1,3-diphenylisobenzofuran (DPBF) are shown in FIG. 5.

Referring to FIG. 4, a gradual decrease in absorbance of 1,3-DPBF at 410 nm under continuous light irradiation was observed in the presence of MeO-BOD (Compound 3) obtained in Synthesis Example 3.

Referring to FIG. 5, a linear photodegradation rate of 1,3-DPBF was observed in the presence of PY-BOD (Compound 1), PH-BOD (Compound 2), MeO-BOD (Compound 3), and DMA-BOD (Compound 4) obtained in Synthesis Examples 1-4.

From this result, the singlet oxygen generation capability of PY-BOD (Compound 1), PH-BOD (Compound 2), MeO-BOD (Compound 3), and DMA-BOD (Compound 4) obtained in Synthesis Examples 1˜4 could be confirmed.

### Analysis Example 2: TEM Images and UV-Vis Absorbance Spectrums of Supramolecules

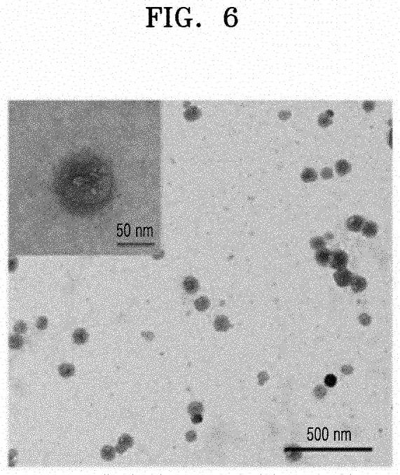

A dimethyl sulfoxide (DMSO) stock solution of MeO-BOD (Compound 3) obtained in Synthesis Example 3 was added dropwise to 3.0 mL of distilled water to obtain 5.0 μM MeO-BOD (Compound 3) supramolecules, followed by a TEM image experiment (200 kV, JEOL-2100F). The results thereof are shown in FIG. 6.

Referring to FIG. 6, the MeO-BOD (Compound 3) supramolecules obtained in Synthesis Example 3 had a diameter of about 72 nm and exhibited a regular spherical morphology.

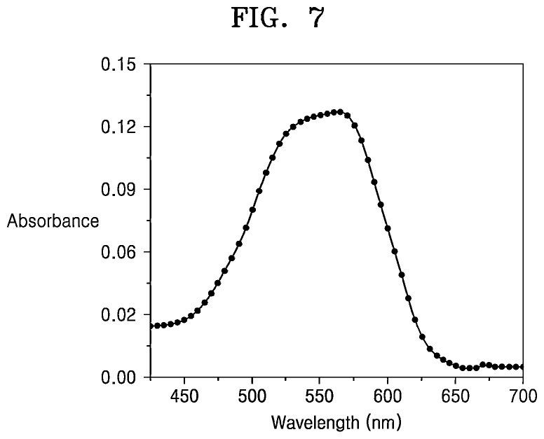

In addition, the MeO-BOD (Compound 3) supramolecules obtained in Synthesis Example 3 were analyzed from UV-vis absorbance spectrums, and the results thereof are shown in FIG. 7.

Referring to FIG. 7, it can be confirmed that in the UV-vis absorption spectrum, the MeO-BOD (Compound 3) supramolecules obtained by Synthesis Example 3 showed a decrease in absorbance over a wide area due to H-aggregation.

### Analysis Example 3: Cell Viability and In Vitro Cell Imaging

(1) In Vitro Cell Imaging Analysis

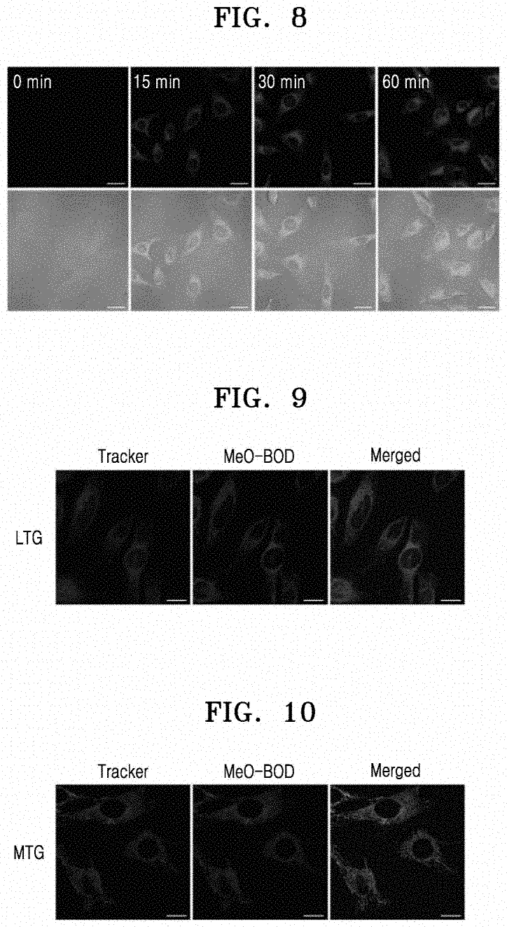

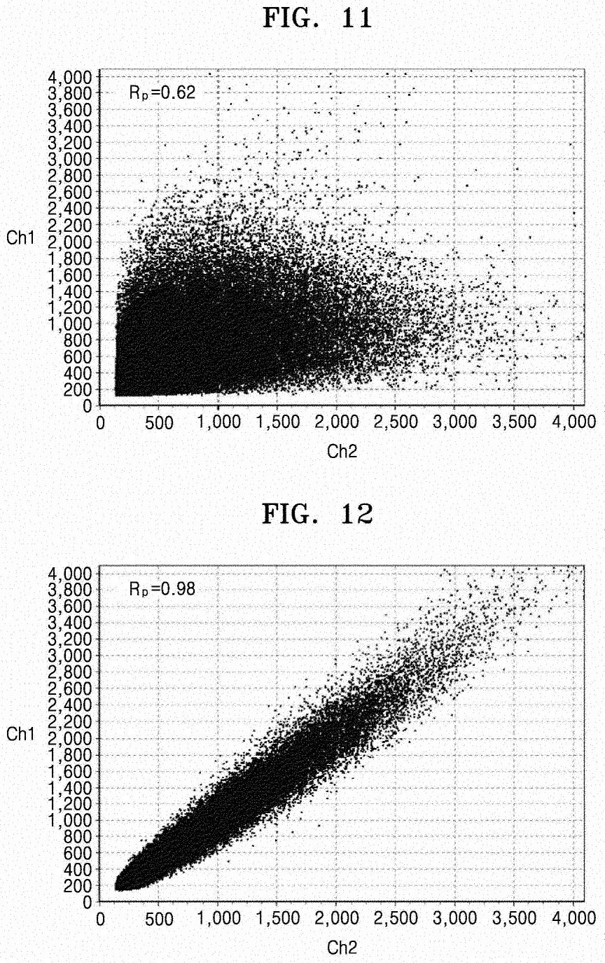

HeLa (human cervix adenocarcinoma) cells were purchased from Korean Cell Line Bank (Seoul, Korea). The cells were grown in Minimum Essential Medium Eagle (MEM) supplemented with 10% fetal serum, 100 U/ml penicillin, and 100 U/ml streptomycin and maintained at 37° C. and 5% CO₂. Cells were seeded in a 35-mm glass bottom dish at a density of 2×10⁵ cells per dish in culture medium. After overnight incubation, HeLa cells were plated on 35-mm confocal dish and incubated for 24 hours. After incubation for 0 minute, 15 minutes, 30 minutes, and 60 minutes with 1.0 μM MeO-BOD (Compound 3) supramolecules obtained in Analysis Example 2 at a concentration of 1.0 μM for 1 hour at 37° C. under 5% CO₂, the cells were further stained with LysoTracker Green DND 26 or/and MitoTracker Green FM (500 nM). Cell imaging was performed with a laser confocal microscope. The results thereof are shown in FIG. 8, FIG. 9, FIG. 10, FIG. 11, and FIG. 12. Here, MeO-BOD (Compound 3) obtained in Synthesis Example 3 had an excitation wavelength of 559 nm, and the excitation wavelength of LysoTracker Green DND 26 (LTG) and MitoTracker Green FM (MTG) was 473 nm. MeO-BOD (Compound 3) obtained in Synthesis Example 3 had an emission wavelength of 575-675 nm and LTG and MTG were sampled at an emission wavelength of 490-540 nm.

Referring to FIG. 8, 1.0 μM MeO-BOD (Compound 3) supramolecules obtained in Analysis Example 2 were rapidly internalized by a live cell and exhibited strong emission within the cell cytoplasm in a fluorescence image. From this result, it could be confirmed that 1.0 μM MeO-BOD (Compound 3) supramolecules obtained in Analysis Example 2 may be used as an imaging-guided photodynamic therapy agent.

Referring to FIG. 9, FIG. 10, FIG. 11, and FIG. 12, It can be seen that 1.0 μM MeO-BOD (Compound 3) supramolecules obtained in Analysis Example 2 have a higher Pearson's coefficient of mitochondria than that of lysosomes. From this result, it could be confirmed that 1.0 μM MeO-BOD (Compound 3) supramolecules obtained in Analysis Example 2 is a mitochondria-targeted photodynamic therapy agent.

(2) Cell Viability (%) Analysis

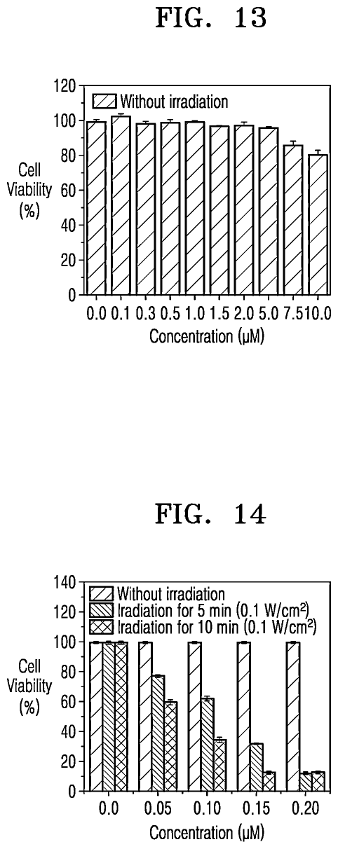

HeLa cells were seeded at 10,000 cells per well in a 96-well plate with 100 μl culture medium, and then cultured at 37° C. for 24 hours under 5% CO₂. 100 μL of MeO-BOD (Compound 3) having a concentration of 0-10.0 μM obtained in Synthesis Example 3 were added to each well and further cultured for 24 hours. Then, cell culture media were replaced with 100 μL of fresh media. Subsequently, in the darkroom, or for different light irradiation times (0, 5, and 10 minutes), the cells were continuously cultured for 24 hours using halogen lamp 560 nm light (0.1 W/cm²). Subsequently, 10 μL of MTT solution (5 mg/mL) and 10 μL of fresh MEM were added to each well for 4 hours, the media were carefully removed, and blue-colored formazan generated by addition of 100 μL of DMSO to each well was dissolved. Then, absorbance at OD 650 nm was recorded using Spectramax Microwell plate reader. Here, cell viability was calculated using Equation 1 below. Calculations were made using the viability of untreated sample cells as 100%, and the data were expressed as the average and standard deviation (SD) from three independent experiments. The results thereof are shown in FIG. 13 and FIG. 14.

Cell viability (%)=(ODₛ−OD_{blank}/OD_{control}−OD_{blank})×100% [Equation 1]

The data were expressed as the average and standard deviation (SD) from three independent experiments.

Referring to FIG. 13 and FIG. 14, MeO-BOD (Compound 3) obtained in Synthesis Example 3 showed negligible cytotoxicity in the dark and showed excellent biocompatibility in cells in vitro. As the concentration of MeO-BOD (Compound 3) obtained in Synthesis Example 3 and 560 nm light irradiation (0.1 W/cm², 5 min or 10 min) increased, the viability of HeLa cells gradually decreased, and even at an extremely low concentration of 0.15 μM, the growth inhibition rate thereof reached about 88%. Half maximal inhibitory concentration (IC₅₀) of MeO-BOD (Compound 3) obtained in Synthesis Example 3 against HeLa cells was as low as 95 nM under 560 nm light irradiation (0.1 W/cm², 10 minutes). Such an extremely low half-maximal inhibitory concentration (IC₅₀) is considered to be attributed to a high ¹O₂ quantum yield and efficient mitochondria-specific ROS (reaction oxygen species) generation upon light irradiation.

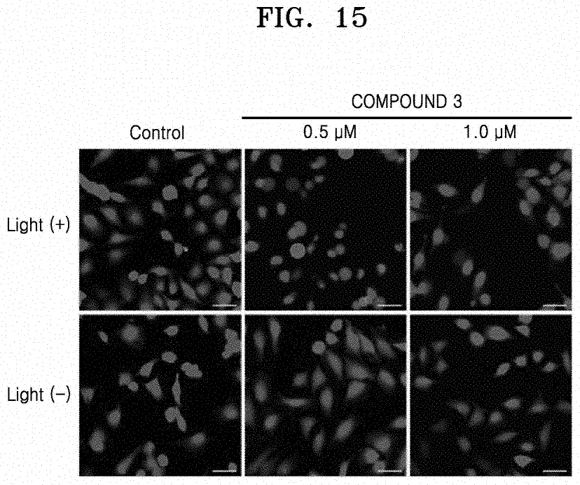

In addition, with respect to HeLa cells cultured with addition of 0.5 μM and 10.0 μM MeO-BOD (Compound 3) obtained in Synthesis Example 3, fluorescence images of calcein AM/PI-stained HeLa cells after irradiation with 560 nm light (0.1 W/cm², 5 minutes or 10 minutes) were analyzed. The results thereof are shown in FIG. 15.

Referring to FIG. 15, as 559 nm laser irradiation time using a confocal laser scanning microscope increased from 0 minute to 10 minutes, it was observed that HeLa cells pre-cultured with MeO-BOD (Compound 3) obtained in Synthesis Example 3 not only had gradually thinning cell membranes, but also showed the formation of numerous blebs (red dots). In contrary, it was observed that the cells unexposed to MeO-BOD of Synthesis Example 3 (control) did not show any obvious change in shape under the same laser irradiation.

### Analysis Example 5: Intracellular Singlet Oxygen Detection Imaging

10 μM of 2,7-dichlorofluorescein diacetate (DCFH-DA) was used as an intracellular singlet oxygen (¹O₂) indicator.

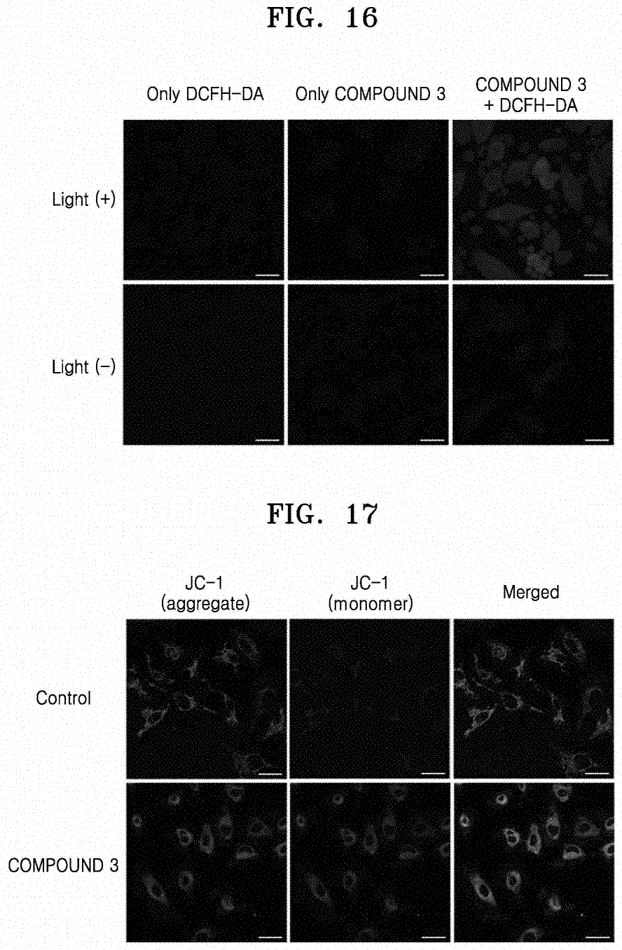

HeLa cells were incubated for 1 hour with 1.0 μM MeO-BOD (Compound 3) supramolecules obtained in Analysis Example 2, and then stained with 10 M DCFH-DA for 30 minutes. Then, after washing with Dulbecco's PBS (DPBS), the HeLa cells were irradiated with halogen lamp 560 nm light (0.1 W/cm²) for 10 minutes. Then, fluorescence images were acquired by confocal laser scanning microscopy to evaluate whether singlet oxygen (¹O₂) were generated inside the cells. The results thereof are shown in FIG. 16. Here, the excitation wavelength was 473 nm and the emission wavelength were collected at 490-540 nm for DCF (dichlorofluorescein).

Referring to FIG. 16, after staining 1.0 μM MeO-BOD (Compound 3) supramolecules obtained in Analysis Example 2 with 10 M DCFH-DA, it could be confirmed that DCFH-DA was oxidized to DCF upon light irradiation (green fluorescence image).

In addition, 2 μM of JC-1 was used as an indicator of fluorescence images of mitochondrial membrane potential of HeLa cells.

JC-1 was added to media, alone, or together with 1 M of MeO-BOD (Compound 3) supramolecules obtained in Analysis Example 2, followed by incubation at 37° C. under 5% CO₂ for 1 hour, and the cells were exposed to 560 nm light irradiation (0.1 W/cm², 10 min) and stained with JC-1 (2M) (Aex=473 nm). As the control, the cells loaded with JC-1 alone by light irradiation were used. The results thereof are shown in FIG. 17. Here, the red channel for aggregates (healthy cells) was 575-675 nm, and the green channel for monomers (apoptotic cells) was 490-540 nm.

Referring to FIG. 17, the control showed strong green fluorescence for live cells, and it can be seen that 1 M MeO-BOD (Compound 3) supramolecules obtained in Analysis Example 2 had low dark-toxicity and excellent biocompatibility. In comparison, almost all of the HeLa cells treated with 1 M MeO-BOD (Compound 3) supramolecules obtained in Analysis Example 2 died, and intense red fluorescence was observed. From this result, it could be confirmed that 1 M MeO-BOD (Compound 3) supramolecules obtained in Analysis Example 2 may be used as a therapeutic agent for cancer treatment in vitro.

1. A compound represented by Formula 1: 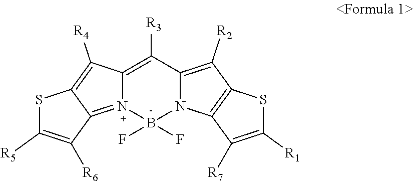
wherein in Formula 1,
R₁ and R₅ are each independently hydrogen, a substituted or unsubstituted C1-C20 alkyl group, a substituted or unsubstituted C6-C30 aryl group, or a combination thereof;
R₂, R₄, R₆, and R₇ are each independently hydrogen, a substituted or unsubstituted C1-C20 alkyl group, or a combination thereof; and
R₃ is a substituted or unsubstituted C6-C30 aryl group, a substituted or unsubstituted C6-C30 heteroaryl group, or a combination thereof.

2. The compound of claim 1,
wherein the substituted or unsubstituted C6-C30 aryl group of R₃ includes a C6-C30 aryl group unsubstituted or substituted with —CHO, —ORₐ, —NRₐ, —NHCORₐ, or —OCORₐ, wherein Rₐ is hydrogen, a C1-C20 alkyl group, or a C6-C20 aryl group.

3. The compound of claim 1,
wherein the substituted or unsubstituted C6-C30 heteroaryl group of R₃ includes a pyridyl group, a pyrrolopyridyl group, a pyrazolopyridyl group, a thienopyridyl group, a pyrimidyl group, a pyrazolyl group, a pyrrolyl group, an imidazolyl group, an indolyl group, an indenyl group, a quinolyl group, or a thiophenyl group.

4. The compound of claim 1,
comprising a compound represented by Formula 2: 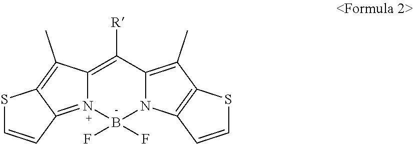
wherein in Formula 2,
R′ is a substituted or unsubstituted C6-C30 aryl group, a substituted or unsubstituted C6-C30 heteroaryl group, or a combination thereof.

5. The compound of claim 4,
wherein the substituted or unsubstituted C6-C30 aryl group of R′ includes a C6-C30 aryl group unsubstituted or substituted with —CHO, —OR′ₐ, —NR′ₐ, —NHCOR′ₐ, —COOR′ₐ, —C₆H₅COOR′ₐ, or —OCOR′ₐ, wherein R′ₐ is hydrogen, a C1-C20 alkyl group, or a C6-C20 aryl group.

6. The compound of claim 1,
comprising Compounds 1 to 4:

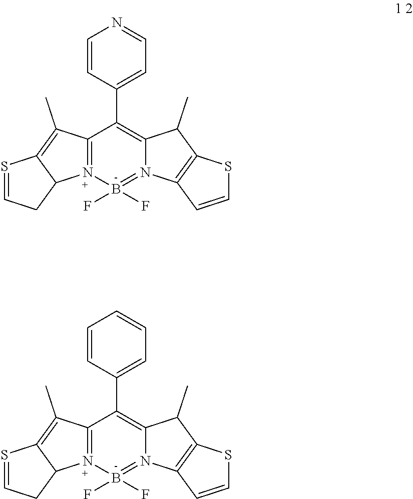

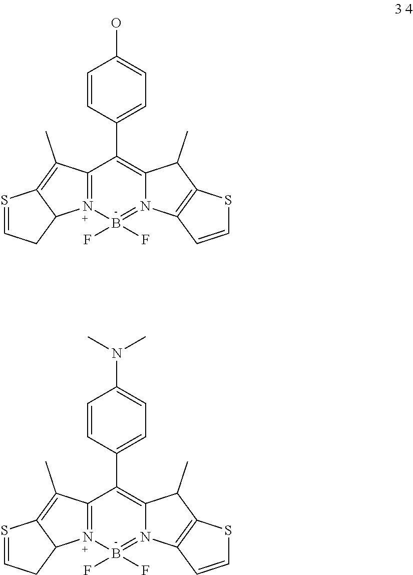

7. A photosensitizer comprising the compound of any one of claims 1 to 6.

8. The photosensitizer of claim 7,
wherein the photosensitizer generates reactive oxygen species when irradiated with light in a wavelength range of 450 nm to 800 nm.

9. The photosensitizer of claim 7,
wherein the photosensitizer is for fluorescence imaging and mitochondria-targeted photodynamic therapy.

10. A composition for mitochondria-targeted diagnosis or treatment of tumors, the composition comprising the compound of any one of claims 1 to 7 or a pharmaceutically acceptable salt thereof as an active ingredient.

11. The composition of claim 10,
wherein the composition comprises the compound or supramolecules of a pharmaceutically acceptable salt thereof.

12. The composition of claim 11,
wherein the supramolecules have a mean size of 1 nm to 200 nm.

13. The composition of claim 10,
wherein the tumors are selected from breast cancer, kidney cancer, testicular cancer, prostate cancer, ovarian cancer, uterine cancer, cervical cancer, vaginal cancer, fallopian tube cancer, rectal cancer, lung cancer, stomach cancer, liver cancer, esophageal cancer, small intestine cancer, pancreatic cancer, oral cancer, melanoma, or sarcoma.

14. A photodynamic therapy method comprising:
contacting a non-human mammalian subject with the composition for diagnosis or treatment of tumors according to claim 10;
allowing time for the composition for diagnosis or treatment of tumors to be distributed within a target cell; and
irradiating a target cell area in the subject with light.
